(19) United States
(12) Patent Application Publication
(10) Pub. No.: US 2008/0242938 A1
(43) Pub. Date: Oct. 2, 2008

(54) Speculum blade covers

(76) Inventor: Daniel Larkin, St. Paul, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/728,755

(22) Filed: Mar. 27, 2007

(51) Int. Cl. A61B 1/00

(52) U.S. Cl. 600/203

(57) ABSTRACT

A disposable speculum blade cover includes a longitudinal body including a proximal end portion spaced from a distal end portion, where the body includes a first wall and an opposing second wall, and the first and second walls coupled to opposing sides of the body to define a pocket configured to receive a speculum blade. Each of the first and second walls extends along a longitudinal axis between the proximal end portion and the distal end portion, and the distal end portion curves away from the longitudinal axis to define a retention lip at a distal end of the cover.

### BACKGROUND

Gynecological examinations have become established as well-care procedures that assist in the early detection of cervical pre-cancerous and cancerous growths. In general, a physician employs a speculum or similar device to expand and support the vaginal vault during the gynecological exam, which enables an unobstructed view of at least the exo-cervical wall.

FIG. 1 is a perspective view of a known speculum as disclosed in Hayes, U.S. Pat. No. 4,807,600. Speculum 20 includes a handle 22 and an adjustment arm 24 coupled to the handle 22 about a hinge 26. A first blade 28 is integrally formed with handle 22, and a second blade 30 is integrally formed with the adjustment arm 24. In the orientation shown in FIG. 1, first blade 28 is a lower blade of the speculum, and second blade 30 is an upper blade. Each of blades 28, 30 include an inner surface 31 and an outer surface 33, where the outer surfaces 33 are those surfaces oriented to contact tissue of the patient. Movement of a thumb piece 34 results in moving upper blade 30 relative to lower blade 28 about hinge 26. A locking device 36 is provided to secure thumb piece 34 in position to maintain a desired position of blades 28, 30 during the gynecological exam.

Other speculum configurations are known, but each generally includes some form of a rigid lower blade and some form of a rigid upper blade. Speculum 20 is commonly provided in stainless steel for easy cleaning, although other rigid speculums are formed of plastic materials. During use, blades 28, 30 are collapsed together and inserted in combination into the vaginal vault. Thumb piece 34 is depressed to separate upper blade 30 from lower blade 28, thus expanding the walls of the vaginal vault. Locking device 36 is engaged to secure blades 28, 30 in their desired position. In this manner, the physician is able to visualize a portion of the cervix and have access to the endo-cervical canal for examination and/or the removal of samples/cells. However, when so positioned, outer surface 33 of upper blade 30 applies pressure along the anterior midline of the pubic symphysis and the apposed internal vaginal walls, which can cause tissue discomfort during the examination procedure.

The use of speculums in gynecological procedures dates back to the Roman period. Improved speculums that are more comfortable will encourage patients to regularly schedule and follow through with these useful gynecological exams. For these and other reasons, there is a need for the present invention.

### SUMMARY

One embodiment provides a disposable speculum blade cover. The speculum blade cover includes a longitudinal body including a proximal end portion spaced from a distal end portion, where the body includes a first wall and an opposing second wall, and the first and second walls coupled to opposing sides of the body to define a pocket configured to receive a speculum blade. Each of the first and second walls extends along a longitudinal axis between the proximal end portion and the distal end portion, and the distal end portion curves away from the longitudinal axis to define a retention lip at a distal end of the cover.

Another embodiment provides a kit of parts configured for attachment to blades of a speculum. The kit of parts includes a first speculum blade cover and a second separate speculum blade cover. Each of the first and second speculum blade covers includes a longitudinal body including a proximal end portion spaced from a distal end portion, and a first wall and an opposing second wall. The first and second walls are coupled to opposing sides of the body to define a pocket configured to receive a speculum blade. Each of the first and second walls extends along a longitudinal axis between the proximal end portion and the distal end portion, and the distal end portion curved away from the first wall and the longitudinal axis to define a retention lip at a distal end of the cover.

Another embodiment provides a disposable speculum blade cover. The speculum blade cover includes a longitudinal body including a proximal end portion spaced from a distal end portion, where the body includes a first wall and an opposing second wall. The first and second walls are coupled to opposing sides of the body to define a pocket configured to receive a speculum blade, and an exterior surface of the second wall comprises a plurality of reticulated pads.

### BRIEF DESCRIPTION OF THE DRAWINGS

Embodiments are better understood with reference to the following drawings. The elements of the drawings are not necessarily to scale relative to each other. Like reference numerals designate corresponding similar parts.

FIG. 1 is a perspective view of a prior art speculum including an upper blade movable relative to a lower blade.

FIG. 2 is a perspective top view of a disposable speculum blade cover according to one embodiment.

FIG. 3 is a perspective bottom view of the speculum blade cover shown in FIG. 2.

FIG. 4A is a magnified view of a pair of opposing inner surfaces of the speculum blade cover taken along line 4-4 of FIG. 3 illustrating textured inner surfaces.

FIG. 4B is a magnified view of another embodiment of textured inner surface of the speculum blade cover.

FIG. 5 is a bottom view of the speculum blade cover shown in FIG. 3 illustrating an internal support structure disposed within a capsule of the cover.

FIG. 6 is a cross-sectional view of the speculum blade cover taken along line 6-6 of FIG. 2 and showing the internal support structure of FIG. 5.

FIG. 7 is a speculum blade cover according to another embodiment.

FIG. 8A is a side view of the speculum blade cover shown in FIG. 2 attached to the upper speculum blade shown in FIG. 1.

FIG. 8B is a side view of another embodiment of a speculum blade cover attached to the upper speculum blade shown in FIG. 1.

FIG. 9 is a perspective view of a kit of parts including two speculum blade covers contained in a package according to one embodiment.

FIG. 10 is a perspective view of a cover assembly for speculum blades including a pair of speculum blade covers and a pair of support walls extending between the pair of speculum blade covers.

FIG. 11A is a cross-sectional view of another embodiment of a speculum blade cover showing an internal support structure having clasp configured to engage with one of the speculum blades of FIG. 1.

FIG. 11B is a cross-sectional view of another speculum blade cover showing another embodiment of an internal support structure.

FIG. 11C is a cross-sectional view of another speculum blade cover showing another embodiment of an internal support structure.

### DETAILED DESCRIPTION

In the following Detailed Description, reference is made to the accompanying drawings which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of the embodiments can be positioned in a number of different orientations, the directional terminology is used for purposes of illustration only and is in no way limiting. As employed in this application, the singular forms “a,” “an,” and “the” include the singular and the plural referents unless the context clearly dictates otherwise. Thus, for example, “a material” includes the specific material and other materials.

It is to be understood that other embodiments may be utilized and structural or logical changes may be made without departing from the scope of the present invention. The following detailed description, therefore, is not to be taken in a limiting sense, and the scope of the present invention is defined by the appended claims.

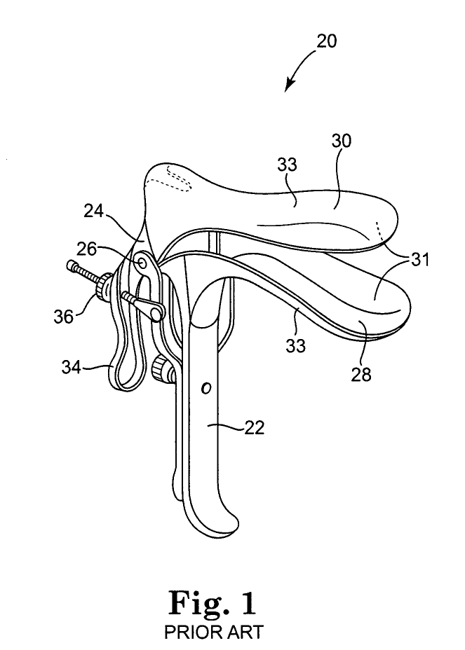

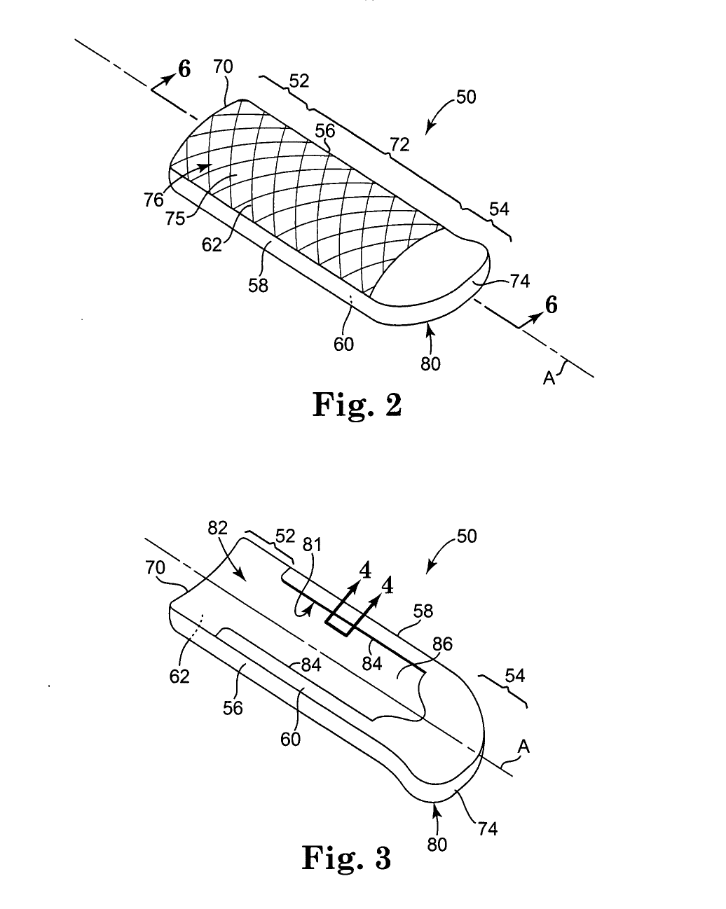

FIG. 2 is a perspective top view of a disposable speculum blade cover 50 according to one embodiment. Speculum blade cover 50 includes a proximal end portion 52 spaced from a distal end portion 54, a longitudinally extending first side 56 generally opposite a longitudinally extending second side 58, and a first wall 60 separated from and coupled to a second wall 62. As illustrated in FIG. 2, opposing sides 56, 58 form opposing lateral sides of cover 50, and walls 60, 62 are generally oriented along a longitudinal axis A. Sides 56, 58 and walls 60, 62 cooperate to define a pocket or cavity (described below with reference to FIG. 3) configured to receive one of speculum blades 28, 30 (FIG. 1).

Proximal end portion 52 extends from a proximal end 70 of cover 50 to a central portion 72. In general, proximal end portion 52 provides an entrance into the pocket or cavity that is formed between first wall 60 and second wall 62. Distal end portion 54 extends between a distal end 74 to the central portion 72. Distal end portion 54 defines a generally compound closed surface formed by the confluence of sides 56, 58 and walls 60, 62 smoothly blending to distal end 74. In one embodiment, sides 56, 58 taper laterally to neck down to a narrower width adjacent distal end portion 54. In other embodiments, for example when cover 50 is configured to cushion a blade of a Graves speculum, sides 56, 58 do not taper.

In one embodiment, walls 60, 62 are configured to complement a shape of the speculum blade 28, 30 to which they are affixed. With additional reference to FIG. 1, one embodiment provides first wall 60 shaped to accommodate the generally concave shape of opposing inner surfaces 31 of speculum blades 28, 30. In one embodiment, second wall 62 is generally convex or curved to accommodate the generally convex outer surfaces 33 of speculum blades 28, 30. In one embodiment, an outer surface 75 of wall 62 includes a reticulated pattern of pads 76. Pads 76 provide comfort pillows, or a padded structure, on an exterior surface of second wall 62. In this regard, pads 76 are preferably soft, resilient, and flexible, and configured to distribute pressure (i.e., “cushion”) wall 62 to protect sensitive tissue during use of cover 50. In one embodiment, an outer surface 75 of wall 62 includes a pad 76 that is soft, resilient, and flexible, and configured to distribute pressure (i.e., “cushion”) wall 62 to protect sensitive tissue during use of cover 50.

In one embodiment, distal end portion 54 curves away from longitudinal axis A in an outward direction to define a retention lip 80 adjacent distal end 74 of cover 50. In other words, retention lip 80 diverges from axis A and curves “up” in the longitudinal direction relative to the orientation of FIG. 2. The generally convex shape of outer surface 75 of second wall 62, in combination with the longitudinally curved distal end portion 54, define a compound curvature on outer surface 75 along distal end portion 54. Retention lip 80 generally retains its curved shape even when removed from one of the speculum blades.

FIG. 3 is a bottom perspective view of speculum blade cover 50 illustrating one embodiment of a pocket 81. In one embodiment, a central portion 82 of first wall 60 is relieved or otherwise removed to define an engagement flange 84 that traverses along sides 56, 58 and distal end 74. Engagement flange 84 is formed as part of first wall 60, which is separated from and offset from an inner surface 86 of second wall 62, in a manner that defines pocket 81. Engagement flange 84 is configured to engage with edges of speculum blades 28, 30 (FIG. 1). In one embodiment, second wall 62 extends continuously between proximal end 70 and distal end 74, and first wall 60 extends between proximal end portion 52 and distal end 74. In particular, in one embodiment second wall 62 is longer than first wall 60.

In one embodiment, engagement flange 84, which is generally disposed along a periphery of cover 50 extends from proximal end portion 52 along each of opposing sides 56, 58 continuously in a U-shape around distal end 74. In this manner, engagement flange 84 is configured to couple about a periphery of one of the speculum blades 28, 30 (FIG. 1). In one embodiment, engagement flange 84 is uniformly sized about a periphery of cover 50, substantially as illustrated in FIG. 3. In another embodiment, an amount of engagement flange 84 that overhangs second wall 62 is non-uniform, such that engagement flange 84 is more pronounced in selected regions of cover 50, e.g., in the region adjacent to distal end 74, such that pocket 81 is “deeper” in the area of retention lip 80, for example. The relieved central portion 82 enables a user to visually observe that the speculum blade (28 or 30) seats properly between second wall 62 and engagement flange 84. In addition, when cover 50 is attached to one of the blades 28, 20 (FIG. 1), engagement flange 84, and in particular the exposed edges of engagement flange 84, is disposed adjacent inner surfaces 31 of the blades 28, 30, i.e., between the blades 28, 30, and away from the patient's tissue, thus minimizing or eliminating the possible pinch-points between cover 50 and the patient.

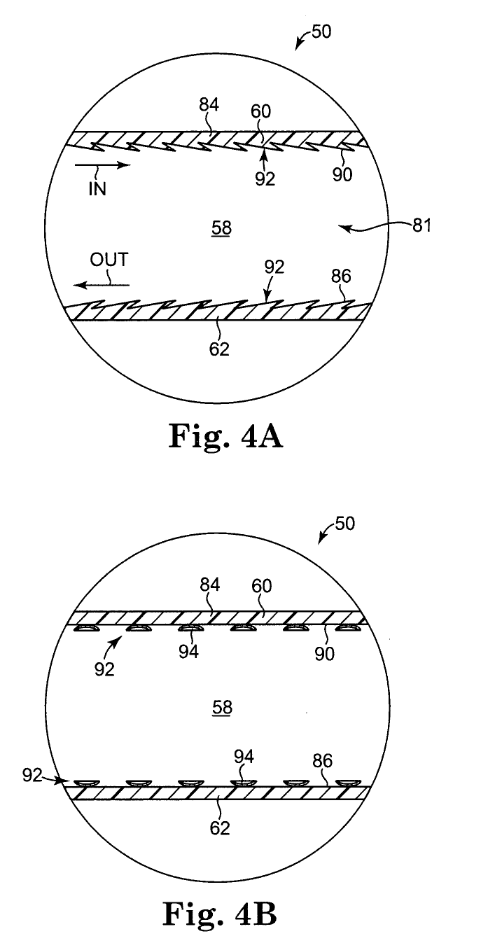

FIG. 4A is a macroscopic view of engagement flange 84 spaced apart from second wall 62 with the view oriented toward side 58. Engagement flange 84 defines an inner flange surface 90 separated from inner surface 86 of second wall 62. In one embodiment, one of inner surface 86 and inner flange surface 90 is structured to define a textured surface 92 having a high degree of sliding friction in one direction, and a low degree of sliding friction in an opposing direction.

For example, in one embodiment textured surface 92 is a saw tooth pattern having peaks oriented to provide minimal sliding friction in the direction of the “IN” arrow, and a high degree of sliding friction in the direction of the “OUT” arrow. One exemplary embodiment provides peaks of textured surface 92 reclined along the direction of the “IN” arrow, although other orientations for textured surface 92 are also acceptable. In this regard, textured surface 92 is a uni-directional structured surface configured to enable the speculum blade 28 or 30 to easily slide into pocket 81 of blade cover 50 along the IN direction, and resist removal of speculum blade 28 or 30 from pocket 81 in the OUT direction. In one embodiment, textured surface 92 is a sharkskin-style of uni-directional structured surface that is smooth in one direction (the IN direction) and characterized by a high friction or roughness in the opposing direction (the OUT direction). In this manner, engagement flange 84 is configured to enable cover 50 to slide easily in place over a speculum blade and yet resist the inadvertent movement of cover 50 relative to the speculum blade during use.

FIG. 4B is a magnified view of another embodiment of textured surface 92 of speculum blade cover 50. Engagement flange 84 defined by wall 60 is spaced apart from second wall 62 with the view oriented toward side 58. In one embodiment, one or both of inner surface 86 and inner flange surface 90 includes suction cups 94. Textured surface 92 of suction cups 94 is configured to “grip” onto an inserted blade 28, 20 (FIG. 1) and provide speculum blade cover 50 with a high friction gripping surface that minimizes the likelihood of speculum blade cover 50 undesirably sliding or moving relative to blades 28, 30 during use.

In one embodiment, suctions cups 94 are integrally formed onto inner surface 86 and inner flange surface 90 when speculum blade cover 50 is fabricated. Suitable materials for suction cups 94 include flexible polymers and/or polymers having high sliding friction (high tack polymers), such as polyurethane, polybutylene, block co-polymers in general, and blends of polyolefin polymers with polyurethane, polybutylene, and block co-polymers. Other suitable polymers are also acceptable for forming suction cups 94.

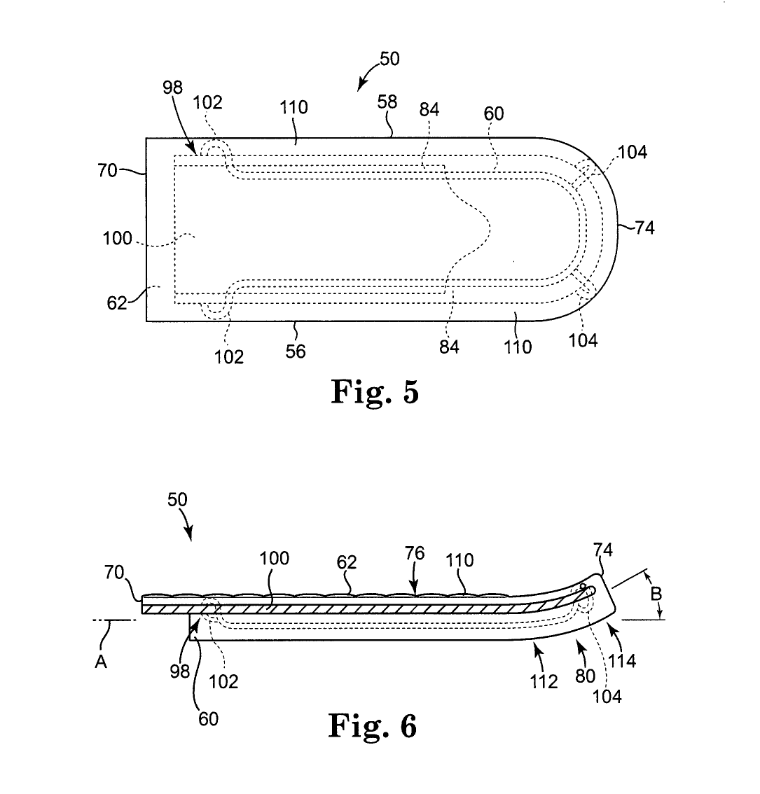

FIG. 5 is a bottom view of speculum blade cover 50 illustrating an internal support structure 98 of cover 50. As noted above, speculum blade cover 50 is configured to retain its shape even when removed from the speculum blades. In one embodiment, internal support structure 98 includes a substantially rigid backbone 100 provided to structurally support second wall 62, and a plurality of ribs 102, 104 provided to structurally support engagement flange 84. To this end, one embodiment provides a self-supporting speculum blade cover 50 including an internal support structure 98. Other embodiments provide a self-supporting speculum blade cover 50 that does not include the plate and ribs, such that sides 56, 58 and walls 60, 62 of cover 50 are molded to be sufficiently rigid and self-supporting without an internal framework.

In one embodiment, backbone 100 includes a reinforcing plate insert. The reinforcing plate insert can be formed of any suitably rigid material, such as plastic or metal. One exemplary embodiment includes backbone 100 insert molded into cover 50 and formed of a polyolefin such as a polyethylene or a polypropylene.

Ribs 102, 104 form an internal portion of engagement flange 84 and couple to rigid backbone 100. In general, ribs 102 are disposed adjacent proximal end 70, and ribs 104 are disposed adjacent distal end 74. Ribs 102 are configured to deflect engagement flange 84 (e.g., flange 84 “angles up” toward wall 62) and ensure contact between flange 84 and inner surface 31 of blade 28 or 30. In this manner, cap 50 is configured to be less likely to “slip” off of speculum blade 28 or 30 during use. Although a pair of ribs 102 and a pair of ribs 104 is illustrated, other configurations for ribs 102, 104 (including fewer ribs or an increased number of ribs) are also acceptable. In combination, backbone 100 and ribs 102, 104 provide structural support for speculum blade cover 50.

In one embodiment, first wall 60, second wall 62, backbone 100, and ribs 102, 104 are integrally molded as a single one-piece unit. Preferably, an exterior portion of walls 60, 62, sides 56, 58, and retention lip 80 is molded of a flexible, durable, and soft capsule 110 over-molded over internal support structure 98 to provide a cushioned surface (e.g., pads 76). Suitable materials for forming capsule 110 include polyolefins in general such as polyethylene or polypropylene, thermoplastic elastomers, or silicone. One suitable material for capsule 110 includes a moldable silicone, such as SILASTIC™ LC-40-2004, a liquid silicone rubber, available from Dow Corning STI, Inc., Kendallville, Ind. In one embodiment, the flexible, durable, and soft capsule 110 is molded from SILASTIC™ silicone having durometer of about 40, although other durometers and materials other than SILASTIC™ silicone are also acceptable. Other suitable materials include silicone and/or silicone rubbers having a lubricious surface, such as, but not limited to, GORE™ SIL-KORE™ materials available from W. L. Gore & Associates, Inc.

FIG. 6 is a cross-sectional view of speculum blade cover 50 taken along line 6-6 of FIG. 2. Capsule 110 cushions internal support structure 98 and includes reticulated pads 76. Capsule 110 generally surrounds speculum blade cover 50 extending over sides 56, 58, walls 60, 62 and retention lip 80. In one embodiment, backbone 100 extends between proximal end 70 and distal end 74 to provide rigidity to retention lip 80.

Retention lip 80 generally includes a first segment 112 that is substantially parallel with longitudinal axis A and a second, end segment 114 that diverges away from longitudinal axis A and wall 60 by an angle B. In one embodiment, second end segment 114 of retention lip 80 diverges away from longitudinal axis A by an angle B of between about 5-45 degrees, and preferably angle B is between about 10 and 30 degrees. In this manner, when cover 50 is placed over one of the speculum blades 28, 30 (FIG. 1), retention lip 80 defines an extending structure that is configured to support the margins of the surrounding deep vaginal wall that approximate the cervical wall, thereby providing improved visualization of the cervix.

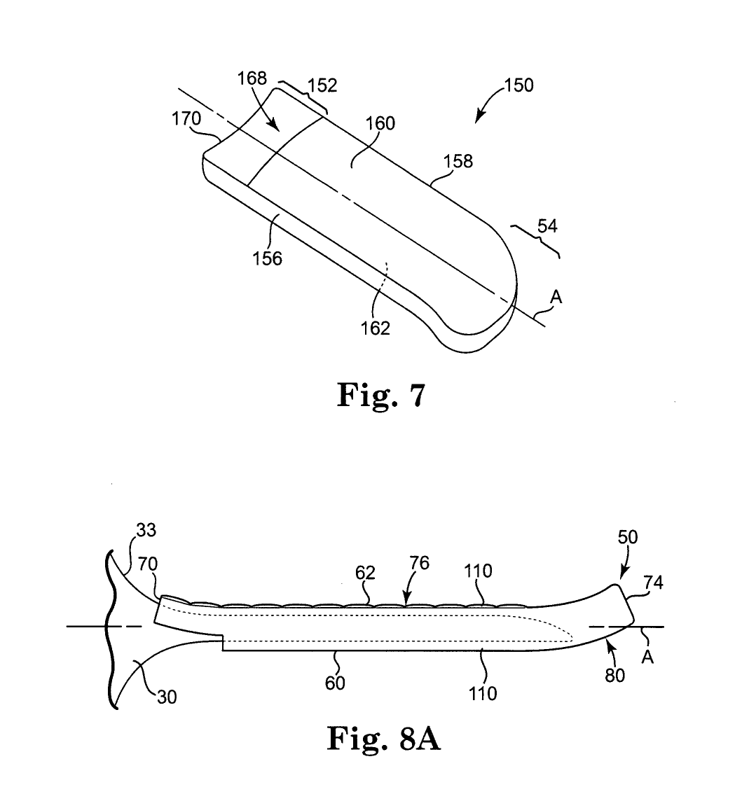

FIG. 7 illustrates a perspective view of another speculum blade cover 150 including a pocket 168 according to another embodiment. Speculum blade cover 150 is similar to cover 50 and includes a proximal end portion 152 separated from a distal end portion 154 extending between opposing sides 156, 158. In one embodiment, first wall 160 is spaced from second wall 162 and extends continuously between opposing sides 156, 158 and end portions 152, 154 to define a pocket 168 having an entrance adjacent proximal end 170 of speculum blade cover 150.

One embodiment provides for an interior surface of one or both of first wall 160 and/or second wall 162 to include the of textured surface 92 described above (e.g., a sharkskin-styled uni-directional texture). In this manner, speculum blade cover 150 can be easily slid over a speculum blade, but resists undesirably sliding off of the speculum blade during a procedure. Generally, speculum blade covers 50, 150 can be removed from a covered speculum blade by being firmly grasped by hand along the sides, followed by the application of manual pressure in forcibly removing the cover 50 or 150 off of the speculum blade.

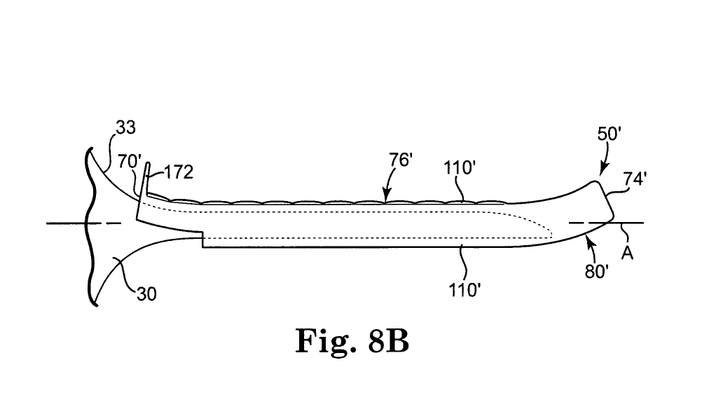

FIG. 8A is a side view illustrating speculum blade cover 50 disposed over speculum blade 30. In one embodiment, wall 62 is longer than wall 60 and is configured to traverse an upper portion of blade 30 along outer surface 33. In this manner, wall 62 extends to a proximal portion of the speculum blade 30 to protectively cushion the pubic symphysis and the apposed internal vaginal walls when the speculum blade 30 and the cover 50 are inserted into the vaginal vault.

Retention lip 80 diverges away from longitudinal axis A to form a structure that is configured to retain a periphery of the exo-cervix. In this regard, although blade 30 is rigid and generally not curved along its length, cushioned cover 50 provides an upswept curvature at the distal end 74 that improves the sight lines along the blade 30 into the vaginal vault. To this end, retention lip 80 is configured to seat along an outer portion of the cervix to provide optimal viewing and comfortable retention of the blade 30 within the vaginal vault. In this manner, speculum blade cover 50 provides improved comfort and improved visualization of the cervix compared to rigid speculum blade 30 alone.

FIG. 8B is a side view of another embodiment of a speculum blade cover 50′ attached to upper speculum blade 30 shown in FIG. 1. Retention lip 80′ diverges away from longitudinal axis A such that cushioned cover 50′ provides an upswept curvature at the distal end 74′ that is configured to improve sight lines along the blade 30 into the vaginal vault for improved visualization of the cervical wall. In addition, cover 50′ includes an upswept skirt 172 adjacent proximal end 70′. Skirt 172 is configured to protect the exterior portions of the vagina from contact and/or pressure from outer surface 33 of blade 30. Skirt 172 functions in a manner similar to a pacifier flange, and protectively prevents outer surface 33 of blade 30 from impinging on sensitive vaginal tissue. Skirt 172 is formed of compliant, durable materials, such as the silicone material described above for capsule 110.

Embodiments of speculum blade cover 50′ provide reticulated pads 76′, soft capsule 110′ over-molded over an internal support structure (not shown), and textured surface 92′ (not shown), as described above in relation to speculum blade cover 50.

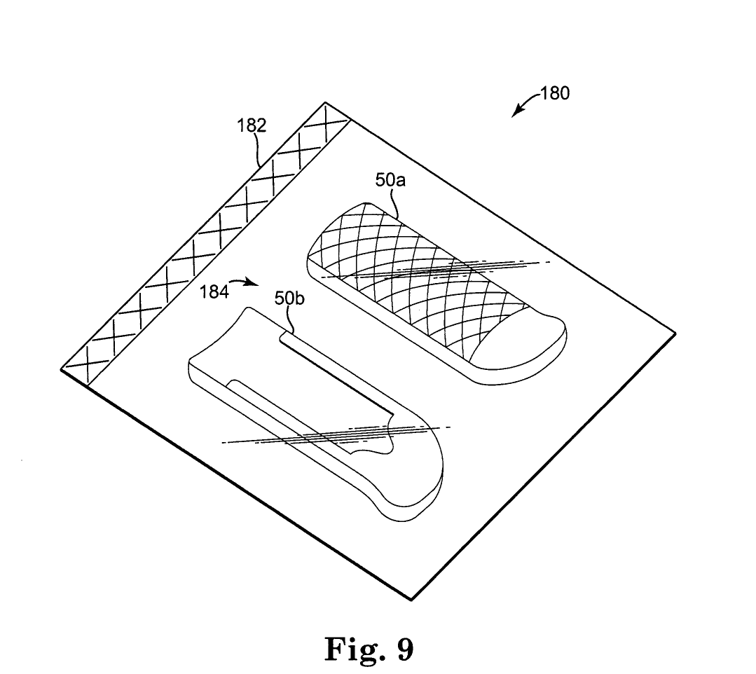

FIG. 9 is a perspective view of a kit of parts 180 according to one embodiment. Kit of parts 180 includes a package 182 containing a pair 184 of speculum blade covers 50a, 50b. Speculum blade covers 50a, 50b are each similar to cover 50 described above, although blade covers 50a, 50b can include a continuous first wall as provided by cover 150.

In one embodiment, each of blade covers 50a, 50b is provided and configured to be slipped over one of the speculum blades 28, 30 (FIG. 1), respectively. In one embodiment, blade cover 50a is identical to blade cover 50b. In another embodiment, blade cover 50a is an upper blade 30 cover having a cushioned second wall 62a that is shorter than the second wall 62b of lower blade 28 cover 50b.

In an exemplary embodiment, and with reference to FIGS. 3 and 6, the space between the first and second walls 60, 62 is configured to receive upper speculum blade 30 such that the second wall 62 extends not more than about 7 cm between the proximal end 70 and the distal end 74. In another exemplary embodiment, the space between the first and second walls 60, 62 is configured to receive lower speculum blade 28 such that the second wall 62 extends not less than about 7 cm between the proximal end 70 and the distal end 74.

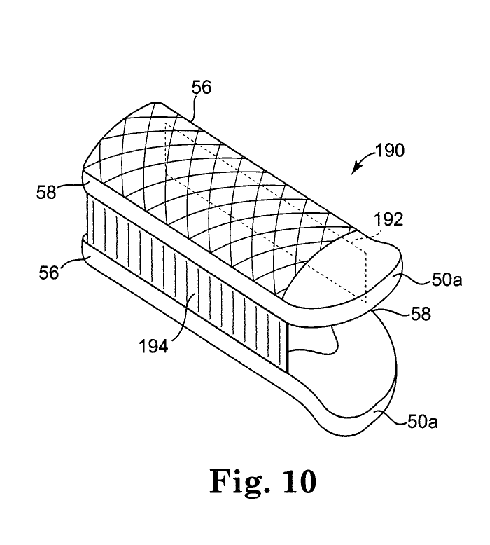

FIG. 10 is a perspective view of another embodiment of a kit of parts including a cover assembly 190 removed from package 182 (FIG. 9). Cover assembly 190 is configured to fit over a set of speculum blades and includes a pair of speculum blade covers 50a, 50b and a pair of support walls 192, 194 extending between the pair of speculum blade covers 50a, 50b. One embodiment of assembly 190 includes support wall 192 coupled to and extending between one set of opposing sides (side 56 and side 58), and support wall 194 coupled to and extending between another set of opposing sides (side 58 and side 56) of covers 50a, 50b, respectively. In one embodiment, support walls 192, 194 are elastic and configured to stretch as speculum blades 30, 28 (FIG. 1) and covers 50a, 50b, respectively, are expanded, and configured to contract and recover their original shape as speculum blades 30, 28 are collapsed.

Cover assembly 190 provides lateral support walls 192, 194 that are configured to restrain lateral walls of the vaginal vault from receding between the open blades 28, 30 of the speculum 20 (FIG. 1). In this manner, cover assembly 190 is configured to provide improved lateral support of the vaginal vault, offering both improved comfort to the patient and improved visualization of the cervix for the physician.

Suitable materials for support walls 192, 194 include plastic in general and thermoplastic elastomers in particular. In one embodiment, support walls 192, 194 are attached to sides 56, 58 by chemical adhesion, mechanical fasteners, or energetic (sonic or thermal) welding. In one embodiment, support walls 192, 194 are formed directly to sides 56, 58 during a molding (fabrication) of covers 50a, 50b and support walls 192, 194.

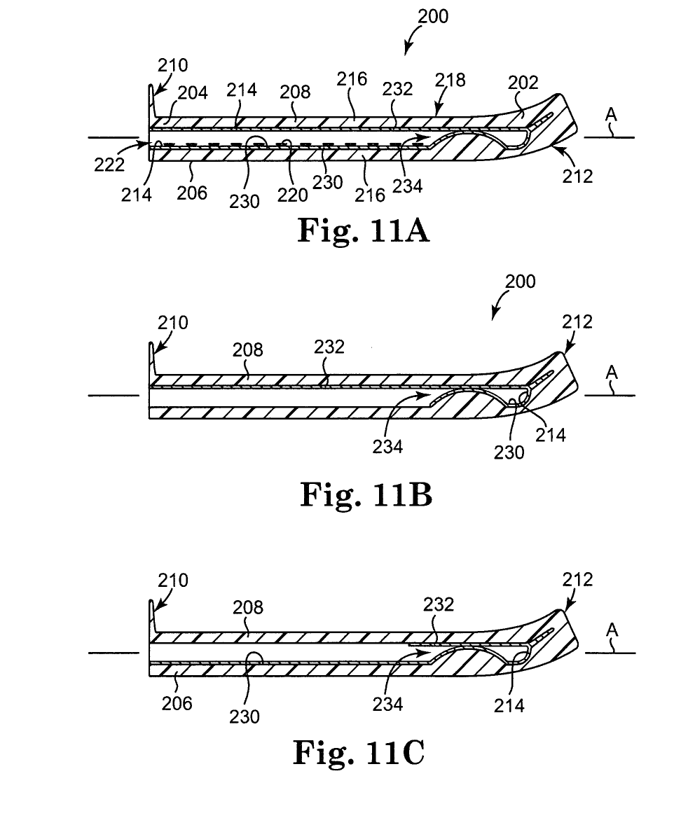

FIG. 11A is a cross-sectional view of another embodiment of a speculum blade cover 200. Speculum blade cover 200 includes a proximal end portion 202 spaced from a distal end portion 204, a first wall 206 separated from and coupled to a second wall 208, a skirt 210 disposed adjacent proximal end portion 202, a retention lip 212 adjacent distal end portion 204, and an internal support structure 214.

Speculum blade cover 200 is similar to the speculum blade covers described above, and embodiments of cover 200 provide a soft capsule 216 encompassing an exterior of cover 200, cushioning pads 218 formed on an exterior surface of wall 208, and a textured surface 220 on at least one interior surface of pocket 222 configured to minimize the risk that cover 200 will slide off of a blade 28, 20 when in use.

Skirt 210 and retention lip 212 generally curve away from longitudinal axis A in an outward direction (upward as oriented in FIG. 11A). In one embodiment, internal support structure 214 reinforces retention lip 212, and each of skirt 210 and retention lip 212 are covered by a soft capsule 216 (e.g., silicone). Reinforced retention lip 212 is sufficiently rigid to provide support to the walls of the vaginal vault, which contributes to improved visualization of the face of the cervix.

In one embodiment, upper and lower bounds of pocket 222 are defined by internal support structure 214, and internal support structure 214 extends substantially between proximal end portion 202 and distal end portion 204 of cover 200. Pocket 222 is configured to enable one of the blades 28, 30 (FIG. 1) of the speculum to slide easily into place within cover 200.

In one embodiment, internal support structure 214 includes a first spine 230 separated from a second spine 232, and spines 230, 232 converge to define a clasp 234 in a region of distal end portion 204. In one embodiment, clasp 234 is configured to frictionally retain a distal portion of one of the blades 28, 30 inserted into pocket 222. The distal portion of the inserted speculum blade 28, 30 is engaged by clasp 234, and retention lip 212 extends distally beyond the distal portion of the inserted speculum blade 28, 30 to provide a semi-rigid lip 212 configured to support portions of the vaginal vault adjacent the exo-cervix.

In one embodiment, internal support structure 214 is formed from a radio-opaque plastic and forms a substantially rigid pocket 222. In another embodiment, internal support structure 214 is formed of a rigid plastic such as polyethylene, or a metal. To this end, pocket 222 enables speculum blade 28, 20 (FIG. 1) to slide easily into cover 200 and be securely engaged by clasp 234.

FIG. 11B is a cross-sectional view of another embodiment of a speculum blade cover 200 showing another embodiment of an internal support structure 214. In this embodiment, lower spine 230 is attenuated in a region of the distal end portion 202 and does not extend to the proximal end portion 204. In particular, first spine 230 is bent to touch second spine 232 and form a portion of clasp 234, and second spine 232 supports wall 208.

FIG. 11C is a cross-sectional view of another embodiment of a speculum blade cover 200. In this embodiment, upper spine 232 is attenuated in a region of the distal end portion 202 and does not extend to the proximal end portion 204. In particular, first spine 230 is bent to touch second spine 232 and to form a portion of clasp 234, and a portion of second spine 232 is removed along wall 208. Second wall 208 is configured to contact outer surface 33 of blades 28, 30 (FIG. 1). Removing a portion of second spine 232 along wall 208 minimizes the possibility that sensitive tissues will be discomforted by internal support structure 214, and yet retention lip 212 is fully supported by internal support structure 214.

The speculum covers described above cushion the rigid blades of speculums. Some embodiments provide for a speculum cover having an upswept distal end that is configured to be seated around a periphery of the cervical wall, providing improved visualization of the cervix. During a gynecological exam, the blades of the conventional speculum (FIG. 1) are expanded one from the other, which can cause uncomfortable pressure to be applied to the local tissue. In addition, the expanded blades experience a resistance from the local tissue that can result in the speculum sliding out from the vaginal vault.

In contrast, embodiments of the speculum covers described above provide a cover that cushions the rigid speculum blades and resist sliding relative to the blades. In addition, the soft, padded blade covers tend to seat the speculum within the vaginal vault to resist movement and sliding of the speculum when positioned for the exam. Other embodiments provide for speculum blade covers that have a textured surface that enables easy placement of the cover over the blade while resisting sliding of the cover off of the blade. This aspect contributes to the padded/covered speculum blade maintaining its desired location throughout the exam.

What is claimed is:

1. A disposable speculum blade cover comprising:
a longitudinal body including a proximal end portion spaced from a distal end portion, the body including a first wall and an opposing second wall, the first and second walls coupled to opposing sides of the body to define a pocket configured to receive a speculum blade, each of the first and second walls extending along a longitudinal axis between the proximal end portion and the distal end portion;
wherein the distal end portion curves away from the longitudinal axis to define a retention lip at a distal end of the cover.

2. The speculum blade cover of claim 1, wherein a central portion of the first wall is removed to define an engagement flange disposed at a periphery of the cover, the engagement flange spaced from the second wall to define the pocket.

3. The speculum blade cover of claim 1, wherein the first wall comprises a continuous first wall extending between opposing sides of the body, the continuous first wall spaced from the second wall to define the pocket.

4. The speculum blade cover of claim 1, wherein the first and second walls each comprise an opposing inner surface defining a texture configured to increase sliding friction of the inner surfaces relative to the speculum blade.

5. The speculum blade cover of claim 4, wherein the texture is one of a uni-directional texture and a suction cup texture.

6. The speculum blade cover of claim 1, wherein the second wall comprises a cushioned pad disposed on an exterior surface.

7. The speculum blade cover of claim 1, further comprising:
an internal support structure over-molded with a polymer capsule that is configured to cushion the internal support structure.

8. The speculum blade cover of claim 7, wherein the internal support structure comprises a substantially rigid plate adjacent to the inner surface of the second wall, the rigid plate extending substantially to the distal end to reinforce the retention lip.

9. The speculum blade cover of claim 8, wherein a central portion of the first wall is removed to define an engagement flange disposed at a periphery of the cover, the body further comprising at least one reinforcement rib coupled between the engagement flange and the rigid plate adjacent to the proximal end portion and at least one reinforcement rib coupled between the engagement flange and the rigid plate adjacent to the distal end portion.

10. The speculum blade cover of claim 7, wherein the internal support structure comprises a first spine and a second spine that converge together to define a clasp that is configured to engage with a blade of the speculum, the first and second spines combining to define an inner pocket surface of the speculum blade cover.

11. The speculum blade cover of claim 1, wherein the proximal end portion of the second wall curves away from the longitudinal axis to define a skirt adjacent a proximal end of the cover.

12. The speculum blade cover of claim 1, wherein the first wall, the engagement flanges, and the opposing sides are integrally formed to define a single continuous piece.

13. A kit of parts configured for attachment to blades of a speculum, the kit of parts comprising:
a first speculum blade cover and a second separate speculum blade cover, each of the first and second speculum blade covers comprising a longitudinal body including a proximal end portion spaced from a distal end portion, a first wall and an opposing second wall, the first and second walls coupled to opposing sides of the body to define a pocket configured to receive a speculum blade, each of the first and second walls extending along a longitudinal axis between the proximal end portion and the distal end portion, the distal end portion curved away from the first wall and the longitudinal axis to define a retention lip at a distal end of the cover.

14. The kit of parts of claim 13, wherein an exterior surface of the second wall of each of the first and second speculum blade covers comprise a plurality of cushioned pads.

15. The kit of parts of claim 13, wherein at least one interior surface of each of the first and second walls comprise a textured surface configured to increase sliding friction of the at least one interior surface relative to a speculum blade inserted into the space between the first and second walls.

16. The kit of parts of claim 13, further comprising:
a cover assembly comprising a first support wall coupled to and extending between one set of the opposing sides of the bodies of the covers and a second support wall coupled to and extending between an other set of the opposing sides of the bodies of the covers.

17. A disposable speculum blade cover comprising:
a longitudinal body including a proximal end portion spaced from a distal end portion, the body including a first wall and an opposing second wall, the first and second walls coupled to opposing sides of the body to define a pocket configured to receive a speculum blade;
wherein an exterior surface of the second wall comprises a plurality of reticulated pads.

18. The speculum blade cover of claim 17, wherein the proximal end portion of the second wall curves away from the longitudinal axis to define a skirt adjacent a proximal end of the cover, and the distal end portion curves away from the longitudinal axis to define a retention lip at a distal end of the cover.

19. The speculum blade cover of claim 17, wherein a central portion of the first wall is relieved between the end portions to define a peripheral engagement flange, the engagement flange offset from the second wall to define the pocket.

20. The speculum blade cover of claim 19, wherein the second wall comprises an inner wall surface separated from an inner flange surface of the engagement flange, at least one of the inner wall surface and the inner flange surface comprising a texture configured to increase sliding friction of the inner surfaces relative to the speculum blade.
